(19) United States
(12) Patent Application Publication
(10) Pub. No.: US 2025/0191179 A1
(43) Pub. Date: Jun. 12, 2025

(54) A MEDICAL IMAGE PROCESSING DEVICE, METHOD AND COMPUTER PROGRAM PRODUCT FOR CONTROLLING DISPLAY OF AN IMAGE FROM A PLURALITY OF IMAGE SOURCES

(71) Applicant: Sony Group Corporation, Tokyo (JP)

(72) Inventors: Jan RENKIN, Philipssite (BE); Junichi YOKOTA, Tokyo (JP)

(21) Appl. No.: 18/844,345

(22) PCT Filed: Mar. 17, 2023
(86) PCT No.: PCT/EP2023/056889

§ 371 Date: Sep. 5, 2024

(30) Foreign Application Priority Data
Mar. 30, 2022 (EP) 22165395.9

(51) Int. Cl. G06T 7/00; G16H 30/40; G16H 40/20; G16H 50/20

(52) U.S. Cl. CPC G06T 7/0012; G16H 30/40; G16H 40/20; G16H 50/20

(57) ABSTRACT

A medical image processing device for controlling display of an image from a plurality of image sources, the medical image processing device comprising processing circuitry configured to: receive a plurality of image signals from a respective plurality of image sources, the plurality of image signals each including image data for display and additional data, wherein at least one the image data and the additional data includes information regarding one or more vital signs of a patient being the target of that image source; generate an assessment for each of the plurality of image signals in accordance with processing performed on at least one of the image data and the additional data of each image signal; and generate a control signal for controlling a display device to display an image comprising image data of at least one of the image signals from the plurality of image sources in accordance with the assessment which has been generated for each of the plurality of image signals.

### BACKGROUND

### Field of the Disclosure

The present invention relates to a medical image processing device, method and computer program product for controlling display of an image from a plurality of image sources.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

Imaging devices are used in a wide range of situations in order to capture images of a scene. These image devices are an example of image sources (being a source which can provide an image to a device such as a server or the like). There are a very wide range of different types of image sources. In a medical environment, such as a hospital or the like, an image source may include an endoscopic imaging device, a microscopic imaging device, a room camera, a surgical camera, a bed camera or the like. There may also be an image source which provides an image of an output of a device. An example of output of a device in a medical environment may be vital information regarding a patient which has been measured by a device. Accordingly, there are a wide range of image sources which can provide an image signal to a server (or other computational device).

These image sources may provide the image signal as a single image or a stream of images. A stream of images may also include a video from the image source. Therefore, image sources may provide a number of different types of images.

Often, there are numerous image sources which provide an image signal to a server (or other computational device) at the same time. This is particularly the case in a large or complex environment, such as a medical environment, where multiple image sources are required at the same time in order to ensure that a medical professional has access to all of the information they require.

In a hospital (as an example of a medical environment) there may be multiple image sources in a single room. In a surgical theatre, for example, there may be image sources including a surgery camera, a microscope imaging device and a heart rate monitor which all provide image signals to a server. Moreover, there may also be multiple image sources across a number of different rooms within the hospital. In a hospital, for example, there may be a number of surgical theatres each providing a number of image sources. In addition, there may also be a number of intensive care units (with each bed in the intensive care unit having a number of image sources, such as a bed camera or the like).

As such, in a large or complex environment (such as a medical environment) a very substantial number of image sources may be capable of providing image signals to a server (or other computational device). The number of image sources providing an image signal can make it very difficult for a medical professional to receive necessary medical information, monitor the progress of an operation room, or ascertain the status of an intensive care unit.

This problem will be exacerbated as the use of image sources (such as imaging devices) becomes even more wide spread within a medical environment.

It is an aim of the present disclosure to address these issues.

### SUMMARY

In a first aspect of the disclosure, a medical image processing device for controlling display of an image from a plurality of image sources is provided, the medical image processing device comprising processing circuitry configured to:

receive a plurality of image signals from a respective plurality of image sources, the plurality of image signals each including image data for display and additional data, wherein at least one the image data and the additional data includes information regarding one or more vital signs of a patient being the target of that image source; generate an assessment for each of the plurality of image signals in accordance with processing performed on at least one of the image data and the additional data of each image signal; and generate a control signal for controlling a display device to display an image comprising image data of at least one of the image signals from the plurality of image sources in accordance with the assessment which has been generated for each of the plurality of image signals.

In a second aspect of the disclosure, a medical image processing method of controlling display of an image from a plurality of image sources is provided, the method comprising: receiving a plurality of image signals from a respective plurality of image sources, the plurality of image signals each including image data for display and additional data, wherein at least one of the image data and the additional data includes information regarding one or more vital signs of a patient being the target of that image source; generating an assessment for each of the plurality of image signals in accordance with processing performed on the at least one of the image data and the additional data of each image signal; and generating a control signal for controlling a display device to display an image comprising image data of at least one of the image signals from the plurality of image sources in accordance with the assessment which has been generated for each of the plurality of image signals.

In a third aspect of the disclosure, a computer program product comprising instructions which, when the instructions are implemented by a computer, cause the computer to perform a medical image processing method of controlling display of an image from a plurality of image sources is provided, the method comprising: receiving a plurality of image signals from a respective plurality of image sources, the plurality of image signals each including image data for display and additional data, wherein at least one of the image data and the additional data includes information regarding one or more vital signs of a patient being the target of that image source; generating an assessment for each of the plurality of image signals in accordance with processing performed on the at least one of the image data and the additional data of each image signal; and generating a control signal for controlling a display device to display an image comprising image data of at least one of the image signals from the plurality of image sources in accordance with the assessment which has been generated for each of the plurality of image signals.

Further embodiments of the disclosure are defined in accordance with the appended claims.

In accordance with embodiments of the disclosure, an improved way of controlling the display of images from a plurality of image sources is provided. This is particularly advantageous in a medical environment (such as a hospital or the like). That is, the embodiments of the disclosure, a computationally efficient way of optimizing the output from a plurality of image sources can be achieved, which may enable a medical professional to quickly and reliably receive necessary medical information, monitor the progress of an operation room, or ascertain the status of an intensive care unit. This can further improve the outcome of medical procedures.

Of course, it will be appreciated that the present disclosure is not limited to these advantageous technical effects. Other advantageous technical effects will become apparent to the skilled person when reading the disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an example apparatus in accordance with embodiments of the disclosure;

FIG. 2A illustrates an example medical environment in accordance with embodiments of the disclosure;

FIG. 2B illustrates an example image display in a medical environment;

FIG. 3 illustrates an example apparatus in accordance with embodiments of the disclosure;

FIG. 4 illustrates an example image processing system in accordance with embodiments of the disclosure;

FIG. 5 illustrates an example process flow in an image processing system in accordance with embodiments of the disclosure;

FIG. 6A illustrates an example image display in a medical environment in accordance with embodiments of the disclosure;

FIG. 6B illustrates an example image display in a medical environment in accordance with embodiments of the disclosure;

FIG. 7 illustrates an example method in accordance with embodiments of the disclosure.

### DESCRIPTION OF THE EMBODIMENTS

Referring now to the drawings, wherein like reference numerals designate identical or corresponding parts throughout the several views.

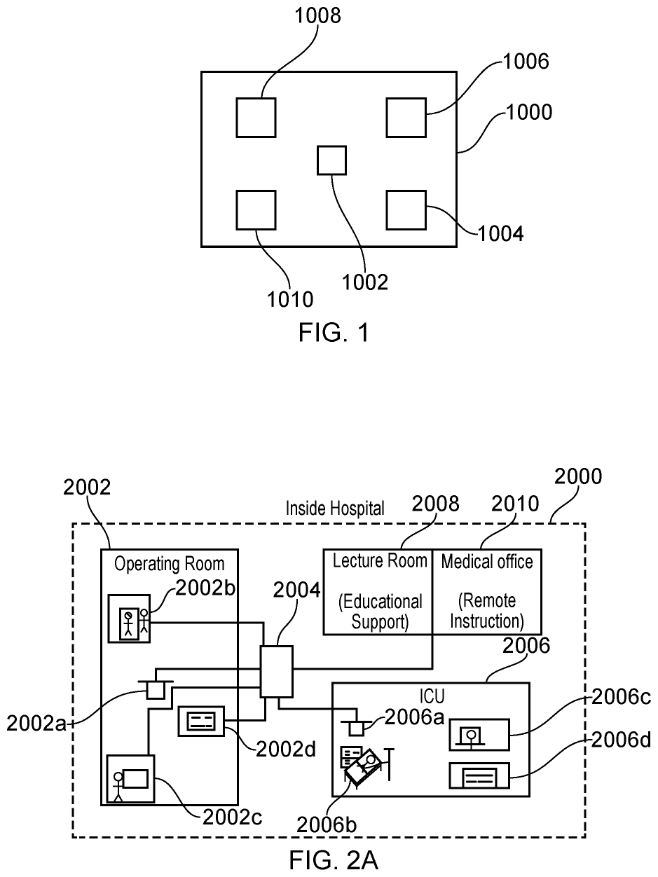

Referring to FIG. 1, an apparatus 1000 according to embodiments of the disclosure is shown. Typically, an apparatus 1000 according to embodiments of the disclosure is a computer device such as a personal computer or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet computing device.

The processing circuitry 1002 may be a microprocessor carrying out computer instructions or may be an Application Specific Integrated Circuit. The computer instructions are stored on storage medium 1004 which maybe a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the processing circuitry 1002. The user input device 1006 may be a touch screen or may be a mouse or stylist type input device. The user input device 1006 may also be a keyboard or any combination of these devices.

A network connection 1008 may optionally be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualise the operation of the system. In addition, the display device 1010 may be a printer, projector or some other device allowing relevant information generated by the apparatus 1000 to be viewed by the user or by a third party.

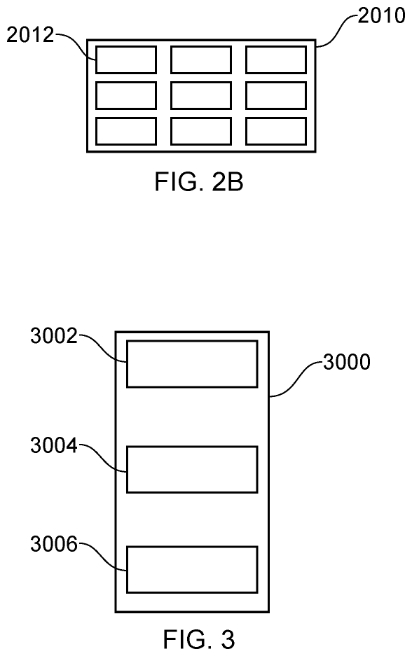

Turning now to FIG. 2A, an example of a medical environment (which is, in this example, a hospital) is shown. Other examples of medical environments include a general practice surgery, a dental practice or the like.

The hospital 2000 comprises a number of different areas. The first of these areas is an operating room 2002. The operating room is also known as an operating theatre or surgical theatre. The operating room is where a medical professional (such as a surgeon) or a team of medical professionals may perform a surgical operation on a patient. The surgical operation may be invasive (such as open surgery) or minimally or non-invasive surgery (such as laparoscopic surgery or the like). Within this operating room 2002, there may be a number of image sources 2002a, 2002b, 2002c, and 2002d.

Image source 2002a is, in this example, a room camera which is configured to capture images of the operating room and provide these images as an image signal to server 2004 (or other computational device). These images from the image source 2002a may therefore provide an overview of the configuration of the operating room 2002.

Image source 2002b is, in this example, a camera which can provide images of the surgical procedure which is being performed on a patient by the medical professional or team of medical professionals. This may include, for example, images from a medical instrument such as a laparoscopic medical imaging device, an endoscopic medical imaging device or the like. The images from the image source 2002b are provided as an image signal to server 2004 (or other computational device).

Image source 2002c is, in this example, an image of a display viewed by a medical professional within the operating room during the surgical operation. This may include, for example, a master console viewed by a medical professional (such as a surgeon) when controlling a robotic surgical device during a surgical procedure.

Image source 2002d is, in this example, the output from a monitoring device which is configured to monitor vital information regarding a patient during the surgical procedure. The vital information regarding the patient may include the patient's vital signs. Examples of vital signs of the patient include the body temperature of the patient, the pulse rate of the patient, the respiration rate of the patient and/or the blood pressure of the patient. In an example, the image source 2002d may therefore be a blood pressure monitor which is configured to provide an indication of the blood pressure of the patient as an image signal to the server 2004 (or other computational device).

Of course, it will be appreciated that the number and type of image sources within the operating room 2002 may be different from that described in this example. Indeed, the configuration of image sources within the operating room 2002 may vary in accordance with the type of surgical procedure or operation which is to be performed in the operating room 2002.

Moreover, while only a single operating room 2002 is described in this example, there may be many more operating rooms 2002 present within the hospital 2000 and each of these operating rooms 2002 may have its own configuration of image sources configured to provide an image signal to the server 2004.

The hospital 2000 may also include an intensive care unit 2006 (ICU). The intensive care unit may be a specialist hospital ward that is adapted such that treatment and monitoring may be provided for a patient who is very ill. Such an ICU may be staffed with certain medical professionals who are specifically trained to use the sophisticated monitoring equipment found in the ICU.

In this example, the ICU 2006 comprises a number of image sources 2006a, 2006b, 2006c and 2006d.

Image source 2006a is, in this example, a room camera which is configured to capture images of the ICU 2006 and provide these images as an image signal to server 2004 (or other computational device). These images from the image source 2006a may therefore provide an overview of the configuration of the ICU 2006.

Image source 2006b is, in this example, a camera which can provide a dedicated image of a patient's bed in the ICU 2006 (a bed camera). The images from the image source 2006b enable medical professionals to closely monitor a patient during the patient's stay in the ICU 2006. The images from the image source 2006b may be provided as an image signal to server 2004.

Image source 2006c and image source 2006d are, in this example, image sources which can provide an image signal from patient monitoring equipment. The patient monitoring equipment may include blood pressure monitors, oxygen saturation monitors, respiration monitors or the like. The information from these image sources may be provided as an image signal to server 2004.

Of course, it will be appreciated that the number and type of image sources within the ICU 2006 may be different from that described in this example. Indeed, the configuration of the image sources within the ICU 2006 may vary in accordance with the specific needs and requirements of the patient who is staying in the ICU 2006.

Moreover, while only a single ICU 2006 is described in this example, there may be many more ICUs 2006 present within the hospital 2000 and each of these ICUs 2006 may have its own configuration of image sources configured to provide an image signal to the server 2004.

Furthermore, while only an operating room 2002 and ICU 2006 have been described in this example, there may be many other locations within the hospital environment which contain image sources configured to provide image signals to the server 2004.

Now, the image signals from the image sources received by the server 2004 (or other computational device) may be distributed to a number of locations within the hospital 2000. This may occur in substantial real time.

A first location within the hospital 2000 to which the image signals from the image sources may be distributed may be a lecture room 2008. The image signals from the image sources may be used in order to provide training or educational support to students or medical professionals. The training may, for example, be training based on a specific surgical procedure which is being performed in operating room 2002.

Image signals from the image sources may also be distributed to a medical office 2010. The medical office may be a location from which medical professionals can provide medical instructions (remote support) to a person in the operating room 2002 or the ICU 2006.

Consider, now, FIG. 2B of the present disclosure. FIG. 2B illustrates an example image display in a medical environment. This may be the display of a display device present within medical office 2010, for example.

The display 2010 of a display device in the medical office 2010 may show a number of image feeds 2012. Each image feed may be a stream of image data received in an image signal from an image source present within one or more operating rooms 2002 and ICUs 2006.

As demonstrated in FIG. 2B of the present disclosure, multiple image feeds may be displayed on the display device. However, the display device may quickly become cluttered and overcrowded, which limits the ability of a person monitoring the display device to receive the necessary information from the image feeds. Indeed, it may not be possible to display the image feeds from all image sources on the display device, further increasing the risk that critical medical information will be missed. A medical professional in the medical office 2010 may therefore not be able to provide appropriate remote support to a medical processional in the operating room 2002 or the ICU 2006.

Of course, the number of image feeds 2012 shown on the display device is not particularly limited to the number shown in the example of FIG. 2B. Moreover, there may be images from a significant number of image sources which cannot be displayed on the display owing to physical or practical limitations on the number of image feeds which can be displayed. Furthermore, it will be appreciated that an image source in accordance with embodiments of the disclosure may also provide a video signal (being a series or number of distinct frames of image data). The image or video signal may also include an audio signal providing audio data which has been captured at the time of image capture by the image source.

Therefore, as demonstrated with reference to FIG. 2B of the present disclosure, owing to the number of different image sources present within the hospital 2000, it can become very difficult for a person monitoring the display 2010 to appropriately monitor the images from the different image sources.

Returning to the specific example of FIG. 2A of the present disclosure, it will be appreciated that in order to provide appropriate training (in lecture room 2008) or appropriate medical instructions (in medical office 2010) it is important that certain images from the image sources are provided on a display device within these locations. However, it will be appreciated that, owing to the number of disparate image sources described above and the number of different rooms and environments within the hospital (such as the number of operating rooms 2002 and the number of ICUs 2006) it can be very difficult to display the images from the image sources within the lecture room 2008 and/or the medical office 2010. The display area of a display device may limited (e.g. there may be a physical limitation on the number of images sources from which image signals can be displayed). Moreover, even if the display area of the display device is increased (or even if the number of display devices is increased) it can become very difficult for a person present in lecture room 2008 or medical office 2010 to appropriately monitor the images from the different image sources.

As such, the quality of medical training and/or medical support which can be provided using images from the image sources can be quite limited. This is exacerbated as the number of image sources increase (as the size of the hospital and/or the number of image sources per location (such as per operating room) increases).

This can make it very difficult for a person (such as a medical professional) to receive necessary medical information, to monitor the progress of an operation or to ascertain the status of an intensive care unit using images from the image sources.

For these reasons (in addition to the reasons described in the Background) a medical image processing device, a medical image processing method, and a computer program product for controlling display of an image from a plurality of image sources are provided in accordance with embodiments of the disclosure.

### <Apparatus>

FIG. 3 of the present disclosure illustrates an example apparatus 3000 in accordance with embodiments of the disclosure. The apparatus 3000 may be a medical image processing device for controlling display of an image from a plurality of image sources.

The medical image processing device 3000 for controlling display of an image from a plurality of image sources, the medical image processing device may comprise processing circuitry including a receiving unit 3002, a generating unit 3004 and a control unit 3006.

The receiving unit 3002 may be configured to receive a plurality of image signals from a respective plurality of image sources, the plurality of image signals each including image data for display and additional data, wherein the image data and/or the additional data includes information regarding one or more vital signs of a patient being the target of that image source;

The generating unit 3004 may be configured to generate an assessment for each of the plurality of image signals in accordance with processing performed on the image data and/or the additional data of each image signal; and

The control unit 3006 may be configured to generate a control signal for controlling a display device to display an image comprising image data of at least one of the image signals from the plurality of image sources in accordance with the assessment which has been generated for each of the plurality of image signals.

In this way, the medical image processing device 3000 may be configured to control the display of images from a plurality of image sources within a medical environment (such as hospital 2000 described with reference to FIG. 2A and FIG. 2B of the present disclosure). The medical image processing device 3000 efficiently optimizes the output from a plurality of image sources and may therefore enable a medical professional to quickly and reliably receive necessary medical information, monitor the progress of an operation in an operating room and/or ascertain the status of an intensive care unit.

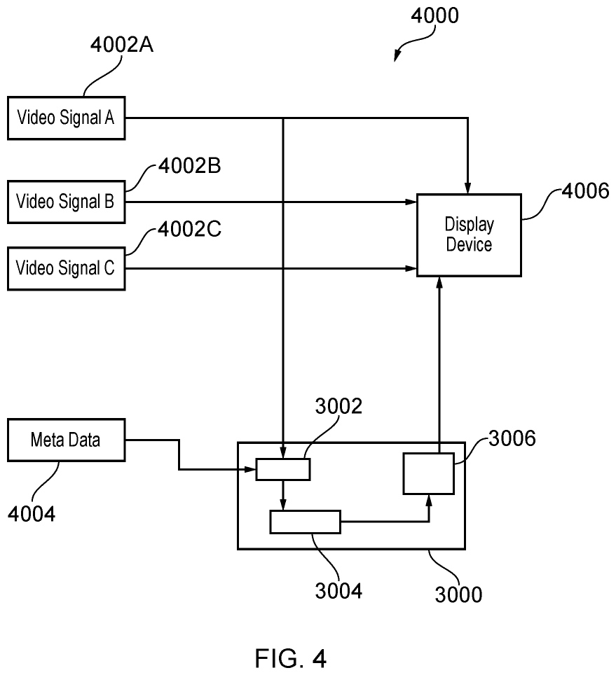

Further details of the medical image processing device 3000 will be described with reference to FIGS. 4, 5, 6A and 6B of the present disclosure.

### <Receiving Unit 3002>

As explained with reference to FIG. 3 of the present disclosure, the medical image processing device may comprise a receiving unit 3002 (or other processing circuitry) configured to receive a plurality of image signals from a respective plurality of image sources.

An image source in accordance with the present disclosure is any device which is able to provide an image signal as output. This may include, for example, types of image capture devices. It may also include computational devices which are able to generate an image signal for output on a display (such as a graphical user interface or the like).

Indeed, an example of a number of different types of image sources which may be located within a medical environment such as a hospital or the like have been described with reference to FIG. 2A of the present disclosure. These can include imaging devices (e.g. room cameras, endoscopic imaging devices, laparoscopic imaging devices or the like), medical monitoring devices (e.g. patient heart rate monitors or the like) or computational devices (such as a device located in operating room 2002 configured to generate a user interface or display which can be used during control of a robotic surgical device). It will be appreciated that these are merely a small number of examples of the very different types of image sources which can be used in accordance with the present disclosure.

Each image source, of the present disclosure, provides an image signal. This image signal includes image data (being an image, or instructions which can be used to create an image, for display). This image data can be provided in any suitable format depending on the type of image source. A room camera (as an example of an image source) may provide images in an image format such as a high definition or 4K image format or the like. Alternatively, a computational device or monitoring equipment (as examples of image sources) may provide information in a format which enable images to be constructed by a display device (e.g. as data (such as patient heart rate) which can be processed by a display device to create an image). The type and format of image data of the image signal from image sources in the present disclosure is not particularly limited in this regard.

The image signal from each image source may also include additional data. This additional data may be data which accompanies the image data and provides certain additional information regarding the image source and/or a target (e.g. a patient) of the image source. Metadata is an example of a type of additional data which can be provided in the present disclosure. This metadata may include information regarding a type of the image source, a location of the image source, a time of image capture, configuration information of the image source, or the like. The additional information may also include contextual information which describes a situation surrounding the images provided by the image source. An example of contextual information is information indicating a type of surgical procedure being performed, a type of illness suffered by a patient on an intensive care unit, a list of medical professionals present during a surgical procedure or the like. Additional data may also include personal information regarding the name of a patient, the age of a patient, patient identification information or the like.

Advantageously, in accordance with embodiments of the disclosure at least one of the image data and the additional data provided in the image signal from an image source may include information regarding one or more vital signs of a patient being the target of that image source. A target of the image source is the patient who is being observed and/or monitored by the image source. In an operating room, the target of the image source is the patient who is undergoing a surgical procedure. In an intensive care unit, the target of the image source is the patient who is being treated on

Here, it will be appreciated that vital signs of a patient include any signs which can provide an indication of the medical status of a patient who is the target of the image source. These vital signs can therefore include the body temperature of the patient, the pulse rate of the patient, the respiration rate of the patient and/or the blood pressure of the patient. Furthermore, the vital signs can include an oxygen saturation level, a level of consciousness of the patient, information regarding a level of pain experienced by a patient, a level of urine output by the patient and the like. Indeed, any information which enables a medical professional to quickly and readily identify to detect and monitor medical problems or conditions (i.e. the medical status of a patient) can be understood as a vital sign of the patient within the present disclosure.

This information regarding one or more vital signs of a patient being the target of the image source can, in accordance with embodiments of the disclosure, be included in either the image data and/or the additional data of the image signal. When the information regarding one or more vital signs of the patient is included in the additional data, said information may be included in the form of a data table or data structure which contains information detailing the vital signs of the patient. Thus, when the information regarding one or more vital signs of the patient is included in the additional information, the vital signs of the patient (such as the patient's blood pressure) can be read directly from the additional information. Alternatively, when the information regarding one or more vital signs of the patient is included in the image data of the image signal, one or more image processing techniques may be applied to the image data in order to extract the information regarding the one or more vital signs of the patient. As an example, it will be appreciated that the image data may include an image of a display of a monitoring device such as a blood pressure monitor. Information regarding the patient's blood pressure may then be extracted from the image data through image processing techniques. Alternatively, for example, the image data may include a picture of the patient. The patient's level of consciousness may then be extracted from the image data through image processing techniques. Therefore, the image data of the image signal from an image source may be used in order to convey information regarding the one or more vital signs of the patient.

Image processing techniques used to extract the information regarding the one or more vital signs of the patient from the image data are not particularly limited in accordance with the present disclosure. These may include use of a trained model. In particular, the image processing techniques may use a machine learning model or a neural network in order to extract the information regarding the one or more vital signs of the patient from the image data.

Consider now FIG. 4 of the present disclosure. FIG. 4 illustrates an example image processing system in accordance with embodiments of the disclosure.

The image processing system 4000 shown in FIG. 4 of the present disclosure may be used in a medical environment such as the hospital 2000 described with reference to FIG. 2 of the present disclosure.

The image processing system 4000 comprises a number of image sources 4002A, 4002B and 4002C, which provide image data to an apparatus 3000 (being an apparatus for controlling display of an image from a plurality of image sources as described with reference to FIG. 3 of the present disclosure. In the context of FIG. 2 of the present disclosure (the hospital environment 2000) the apparatus 3000 may be implemented in the form of the server 2004.

The image data from each of the image sources 4002A, 4002B and 4002C is provided to the apparatus 3000. In particular, apparatus 3000 may comprise a receiving unit 3002 which is configured to receive the plurality of image signals from a respective plurality of image sources. The receiving unit may, in some examples, be a network connection 1008 (such as a connection to a Local Area Network or a Wide Area Network such as the Internet or a Virtual Private Network or the like). Indeed, the receiving unit 3002 of apparatus 3000 may receive the image data from the plurality of image sources 4002A, 4002B and 4002C via any wired or wireless connection.

In some examples, the receiving unit 3002 may be configured to receive 4K High-quality Low-latency transmission from the video sources over a network. Indeed, in some examples, the video sources 4002A, 4002B and 4002C (and more generally any image source in accordance with the present disclosure) may be an Internet Protocol camera (IP camera) which is able to transmit data over a network such as the internet. The receiving unit 3002 may then be configured as a IP receiver which is able to receive such data transmitted over the network from the video sources.

In some examples, the image data from the video sources 4002A, 4002B and 4002C may be encrypted data which can be decrypted by the receiving unit 3002.

In this way, the receiving unit 3002 of apparatus 3000 can receive the image data from the video sources (image sources) 4002A, 4002B and 4002C.

In this example, a separate device 4004 is provided in the image processing system 4000 which generates metadata (as an example of additional data) associated with the image data from each of the video sources 4002A, 4002B and 4002C. This metadata generated by the metadata generation unit 4004 may be information regarding a type or location of each of the video sources 4002A, 4002B and 4002C. The metadata generated by the metadata generation unit may be received by receiving unit 3002 over any wired or wireless network connection in the same manner as described for the image data from the video sources 4002A, 4002B and 4002C. The receiving unit 3002 can then combine the metadata with the image data from each of the video sources 4002A, 4002B and 4002C (using, for example, identification information such as a unique ID for each of the video sources) in order to construct the video signal for each of the video sources (being both the image data and the meta data for each video source).

Alternatively, in some examples, the metadata (or any other type of additional data) may be received by receiving unit 3002 directly from each video source 4002A, 4002B and 4002C with the corresponding image data.

In this way, apparatus 3000 is configured to receive image signals from a plurality of image sources in accordance with embodiments of the disclosure.

### <Generating Unit 3004>

As explained with reference to FIG. 2 of the present disclosure, apparatus 3000 comprises a generating unit 3004 which is configured to generate an assessment of each of the plurality of image signals in accordance with processing performed on the image data and/or the additional date of each image signal.

The assessment generated by the generating unit 3004 of apparatus 3000 is generated in order that the different image signals from the different image sources can be compared. This enables the most important image signals to be extracted from amongst all the image signals from all the image sources which are available.

In some examples, the assessment generated by the generating unit 3004 of apparatus 3000 may be in the form of a value or a score indicative of the relative importance of the image signal in comparison to the other image signals which have been received. By generating such a value or score and assigning said value or score to each image signal, the different image signals can be quickly and efficiently compared.

There are a number of ways in which the generating unit 3004 may generate an assessment for each image signal in accordance with embodiments of the disclosure.

In a first example, the generating unit 3004 may perform processing on the image data and/or the additional data of each image signal in order to determine whether the image signal from each image source is active or inactive. The assessment for each image signal may then indicate whether or not the image signal is an active image signal.

In some examples, an image signal from an image source may be considered to be inactive if the image data of the image signal does not contain any image information. This may be the case, for example, if the image source is connected to the network (such that an image signal is received from the image source) but no images are actually being captured by the image source. This may also happen if an image source is located in a room (such as an operating room 2002 as described with reference to FIG. 2 of the present disclosure) but that room is empty or unoccupied and the lights in that room have been turned off. Alternatively, the image signal may contain no image information if the image source is in a standby mode or the like.

In all these cases, the image signal from the image source may be considered to be inactive.

Alternatively, in some examples, an image signal from an image source may still be determined to be an inactive image signal even if the image data of that image signal contains image information. That is, in some examples, the generating unit 3004 of apparatus 3000 may determine whether the image signal from each image source is active or inactive is based on a level of motion detected in the image data. If there is significant motion or movement detected between different image frames of image data received from an image source, then the generating unit 3004 may identify that the image signal is active. Significant motion or movement may be any level of motion or movement which exceeds a predetermined threshold level of motion or movement (with such predetermined threshold being set in accordance with a situation to which the embodiments of the disclosure are applied).

Alternatively, the generating unit 3004 may be configured to determine whether or not an image signal is active based on one or more of a type of an action detected in the image data (e.g. whether a patient on an ICU is asleep or whether a patient on an ICU is receiving emergency care), a type of person detected in the image data (e.g. whether cleaning or support staff are present in an operating room 2002 or whether a medical professional (such as a surgeon) is present in the operating room), information regarding a noise level determined from the additional data (e.g. if there is noise above a predetermined threshold (such as an alarm) then the image signal may be identified as an active image signal), schedule information determined from the additional data (e.g. whether an operating room 2002 is scheduled to be in use or empty at a given time, with image sources in an operating room which is scheduled to be in use being identified as image sources producing an active image signal) and/or the vital signs of a patient being the target of that image source (e.g. if there is a rapid change in the vital signs of a patient then the image signal may be identified as an active image signal).

Of course, it will be appreciated that there are many other examples of an active image signal or an inactive image signal. Indeed, the way in which the generating unit 3004 of apparatus 3000 decides whether or not an image signal is an active image signal may vary in accordance with the situation to which the embodiments of the disclosure are applied.

In some examples, generating unit 3004 may use a trained model in order to determine whether or not an image signal is an active image signal. The model may be trained on accumulated historical data and/or simulated data in order that the trained model can reliably and accurately identify when an image signal is an active image signal. Artificial intelligence (AI) may thus be used in order to detect whether or not an image signal is an active image signal. This can include machine learning or neural network modes.

In this way, the generating unit 3004 of apparatus 3000 may generate an assessment for each image signal indicating whether or not an image signal is an active image signal. Image signals which are identified as inactive may then be excluded from presentation to the medical professional (with only active signals, or a subset thereof, being provided to the medical professionals).

In a second example, the processing performed by the generating unit 3004 of apparatus 3000 on the image data and/or the additional data of each image signal is processing to determine the priority of the image signal from each image source. The assessment for each image signal may then indicate the priority level of each image signal.

Indeed, in some examples, the priority of the image signal from each image source is determined based on the information regarding one or more vital signs of the patient being the target of that image source. As previously explained the vital signs of a patient may include, for example, the body temperature of the patient, the pulse rate of the patient, the respiration rate of the patient and/or the blood pressure of the patient.

The priority of the image signal from each image source may be determined based on the absolute value of the vital signs of the patient. For example, image signals related to image sources targeting a patient whose blood pressure is either below or above a known range of standard blood pressure values (i.e. a patient who has a critical blood pressure reading) may be given a high priority level.

Alternatively, the priority of the image signal from each image source may be determined based on a rate of change of a value of the vital sign of the patient. For example, image signals related to image sources targeting a patient who is experiencing a rapid decrease in oxygen saturation levels may be given a high priority level.

Conversely, the image signal from an image source targeting a patient who's vital signs are in a normal range and/or which remain stable may be given a lower priority level.

However, the present disclosure is not particularly limited in this regard, and one or more additional factors may also be used in order to ascertain the priority level of an image signal from an image source. Indeed, the priority level may also be determined in accordance with contextual information determined from at least one of the image data and/or additional data of the image signal from an image source.

For example, the priority level of the image signal from each image source located in an operation room may be determined in accordance with a level of skill of medical staff performing a medical operation (with a higher priority given to a more junior member of medical staff), a level of experience of medical staff performing a medical operation (e.g. a lower priority may be given to an image signal related to a surgeon who has successfully performed a specific medical operation predetermined a number of times), a type of surgery being performed (with a more complex or risky surgical procedure being given a higher priority level) and/or a duration of a surgery being performed (e.g. an image signal related to a surgical procedure which exceeds a given period of time (being either an absolute period of time, or an expected period of time for a given type of surgical procedure) may be identified as a higher priority image signal).

Alternatively, the priority level of the image signal from each image source located in an intensive care unit may be determined in accordance with whether a bed is used or not (with image signals relating to a bed which is not occupied being given a lower priority level), a level of skill of medical staff caring for the patient (with image signals relating to a patient being treated by a more junior member of medical staff being given a higher priority level), a level of experience of medical staff caring for the patient (with medical staff caring for a patient who have performed that type of care a number of times before being given a lower priority level), a characteristic of the patient (e.g. an image signal related to an older or more vulnerable patient being given a higher priority level), a type of disease suffered by the patient (with image signals related to a patient suffering from a more dangerous disease being given a higher priority level), and/or a type of injury suffered by the patient (with image signals related to a patient who has a more serious type of injury (such as a head injury) being given a higher priority level).

Of course, there are many different examples of the types of contextual information which can affect the priority level of the image signal generated by the generating unit 3004 and included in the assessment for each of the plurality of image signals. The manner by which the priority level is generated may therefore vary in accordance with the context of the situation to which the embodiments of the present disclosure are applied.

In some examples, generating unit 3004 may use a trained model in order to determine the priority level of an image signal. The model may be trained on accumulated historical data and/or simulated data in order that the trained model can reliably and accurately identify the priority level of an image signal. Artificial intelligence (AI) may thus be used in order to detect whether the priority level of an image signal. This can include machine learning or neural network modes.

In this way, the generating unit 3004 of apparatus 3000 may generate an assessment for each image signal indicating the priority level of an image signal. Image signals which are identified as having a lower priority (such as a priority level below a predetermined threshold level) may then be excluded from presentation to the medical professional (with only higher priority image signals, or a subset thereof, being provided to the medical professionals).

Returning now to FIG. 4 of the present disclosure, it will therefore be appreciated that the image signals received from the video sources 4002A, 4002B and 4002C by receiving unit 3002 of apparatus 3000 may be processed by the generating unit 3004 in order to generate an assessment of for each of the image signals (with that assessment including, for example, an indication as to whether the image signal is active or inactive, and/or the priority level of the image signal).

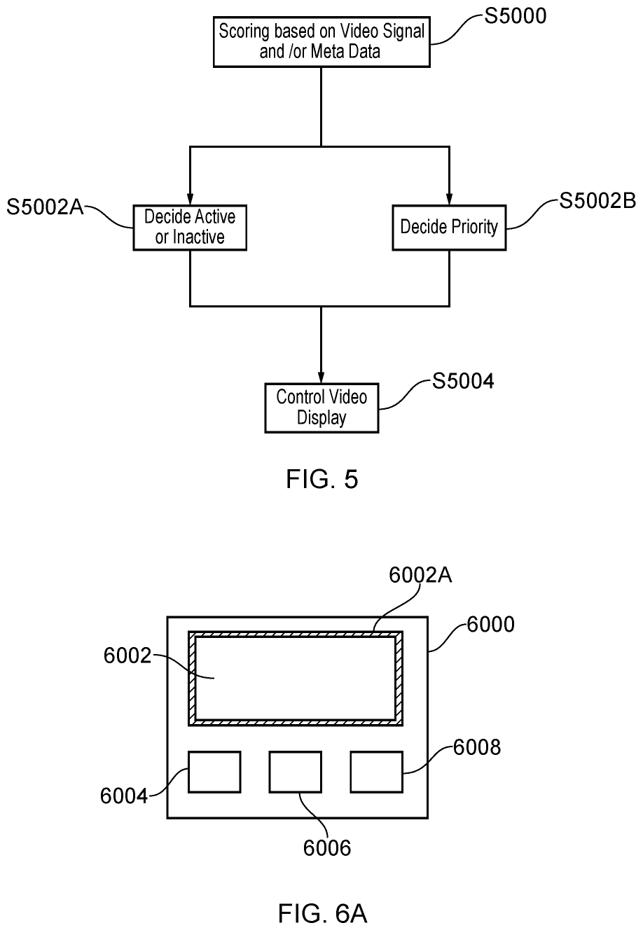

In some examples, the generating unit 3004 may perform a series of processing steps as illustrated in FIG. 5 of the present disclosure. That is, FIG. 5 of the present disclosure illustrates an example process flow in an image processing system in accordance with embodiments of the present disclosure.

Firstly, the generating unit 3004 may obtain the plurality of image signals which have been received by the receiving unit 3002 of the apparatus 3000. Then, in step S5000 of FIG. 5, the generating unit 3004 may generate a score for each of signals from video sources 4002A, 4002B and 4002C of the system 4000. The score may be generated based on the video signal (i.e. the image data or image content) and/or the metadata (or any other type of additional data received for each of the plurality of image signals). Said score may be generated based on the factors which have been described above, including the one or more vital signs of the patient being the target of that video source.

Once the score has been generated, that score can be used by the generating unit 3004 in step S5002A of FIG. 5 in order to decide whether the image signal is an active or inactive image signal. Alternatively, or in addition, that score may also be used in order to decide a priority level of the image signal.

The generating unit 3004 of apparatus 3000 can then generate the assessment information for each image signal (which is to be provided to control unit 3006 of apparatus 3000) such that the assessment information can be used in order to control a display to efficiently and reliably display a selected portion of image signals from the plurality of image sources.

Of course, whether an image signal is an active or inactive image signal and the priority level of the image signal are merely two examples of the type of information which can be included in the assessment generated by the generating unit 3004 for each of the image signals. Indeed, while described as separate examples, it will be appreciated that the generating unit 3004 may include information regarding whether the image signal is an active image signal or not and information regarding the priority level of the image signal within the assessment information for each image signal. Moreover, the present disclosure is not particularly limited in this regard and the generating unit 3004 may generate an assessment for each image signal in any way suitable depending on the context of the situation to which the embodiments of the present disclosure are applied.

### <Control Unit 3006>

As explained with reference to FIG. 3 of the present disclosure, apparatus 3000 may also include a control unit 3006 configured to generate a control signal for controlling a display device to display an image comprising image data of at least one of the image signals from the plurality of image sources in accordance with the assessment which has been generated for each of the plurality of image signals.

In other words, the control unit 3006 can select at least a portion of the image signals which are received from the plurality of image sources as a portion of the image signals which comprise the most important and most relevant medical information to enable a medical professional to monitor the progress of an operation room and/or ascertain the status of an intensive care unit (this may be, for example, a medical professional located in the medical office 2010 described with reference to FIG. 2 of the present disclosure).

Consider the image processing system 4000 of FIG. 4 of the present disclosure. In this example, the control unit 3006 of the medical image processing device 3000 may receive the assessment for each of the plurality of image signals from the generating unit 3004. The control unit 3006 may then use this assessment information for each of the image signals in order to select a portion of the image signals (being the highest priority and/or most active image signals) for display to a medical professional. The control unit 3006 may then, on this basis, generate a control signal for controlling a display device 4006 to display an image comprising image data of at least one of the image signals from the plurality of image sources in accordance with the assessment which has been generated for each of the plurality of image signals. This control signal may then be provided to the display device 4006 by control unit 3006 (or other processing circuitry of the apparatus 3000).

Notably, in the example of FIG. 4 of the present disclosure, the control signal generated by the control unit 3006 of apparatus 3000 is a signal which informs the display device the image signals from which the image data should be displayed and/or how that image data should be displayed. It does not, however, comprise the image signals or image data to be displayed. Instead, the display device 4006 may receive the image signals comprising the image data to be displayed directly from the video sources 4002A, 4002B and 4002C. The display device may then use the control signal received from the control unit 3006 in order to determine which portion of the data received from the video sources should be displayed and, furthermore, how this data received from the video sources should be displayed.

It will be appreciated that the present disclosure is not particularly limited in this regard. In alternative examples, the control unit 3006 may further be configured in order to transmit the selected portion of the image data of the image signals to the display device 4006 for display in accordance with or as part of the control signal. That is, the control signal may instead be a self-contained signal which provides all the necessary information in order to control the display device to display an image comprising image data of at least one of the image signals from the plurality of image sources in accordance with the assessment which has been generated for each of the plurality of image signals.

In some examples, the control unit 3006 may receive configuration information from the display device 4006 regarding the display capabilities of that display device 4006 (such as the size of the display device, the resolution of the display device and/or the data formats capable of being displayed by the display device). In these examples, the control unit 3006 may then adapt the control signal which is generated for the display device in accordance with this configuration information in order to optimize the image displayed on the display device. Indeed, in some examples where there are a plurality of display devices (such as a display device in lecture room 2008 and a display device in medical office 2010) the control unit 3006 may generate the control signal such that the display of the images is optimized for each independent display device respectively.

Of course, it will be appreciated that the control unit 3006 may further be configured in order to repeatedly or periodically generate a control signal for the display device 4006 in order that a continual display of images is provided on the display device 4006. The control signal may be generated at a fixed frequency (corresponding to a frequency of the display device 4006) or, alternatively, the control signal may be updated at regular time intervals (such that the image shown on the display device is regularly updated and refreshed) and/or may be dynamically generated in accordance with the reception of image/video signals from the respective image/video sources.

In this way, control unit 3006 of apparatus 3006 performs step S5004 of FIG. 5 of the present disclosure by generating a control signal for controlling a display device to display an image comprising image data of at least one of the image signals from the plurality of image sources in accordance with the assessment which has been generated for each of the plurality of image signals.

Of course, the actual image data of the plurality of image sources which is selected for display is not particularly limited in accordance with embodiments of the disclosure. Indeed, it may vary depending on the type of assessment information generated by generating unit 3004 of apparatus 3000—including, for example, whether that assessment information indicates whether an image signal is active or inactive and whether that assessment information indicates a priority level of the image signal.

Concerning the activity level of the image signal, in some examples of the present disclosure, control unit 3006 of apparatus 3000 may be configured to generate a control signal configured for controlling the display device to display an image comprising image data from a given image source when the image signal from that image source is active. In other words, the control signal may indicate a selected portion of the image signals from the plurality of image sources which are active image signals. Display of less relevant image data from image sources whose image signals have been identified as inactive may therefore be suppressed. In this way, the most relevant image data can quickly and efficiently be selected for display to the medical professional.

Alternatively, or in addition, in some examples of the disclosure, control unit 3006 of apparatus 3000 may be configured to generate a control signal configured for controlling the display device to display an image comprising image data from a given image source when the image signal from that image source has been identified as being a high priority image signal (based on the assessment information generated by generating unit 3004 of apparatus 3000, for example). Display of less relevant image data from image sources whose image signals whose image signals have been identified as lower priority may therefore be suppressed. In this way, the most relevant image data can be quickly and efficiently be selected for display to the medical professional.

Indeed, these factors (activity level and priority) may also be combined by control unit 3006 when generating the control signal for the display device 4006, such that only the highest priority active signals are displayed to the medical professional. This may be particularly efficient in ensuring that the most relevant image data can be selected for display to the medical professional.

In some examples, the control signal generated by control unit 3006 of apparatus 3000 may be configured such that image data from only the highest priority, most active image signal may be displayed to the medical professional. In other examples, the image data may come from a plurality of image sources (with such a plurality of image sources including at least a portion of the image sources from which an image signal has been received).

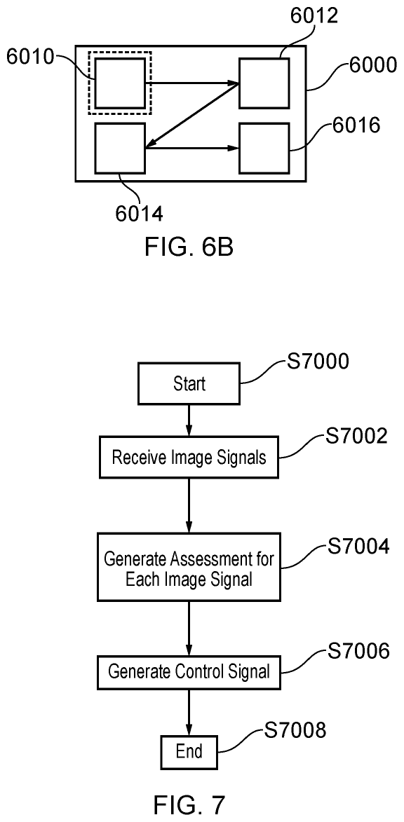

Consider now FIG. 6A of the present disclosure. FIG. 6A of the present disclosure illustrates an example image display in a medical environment in accordance with embodiments of the disclosure.

A display screen 6000 of a display device is shown in FIG. 6A. The display screen may be a display (either internal or external) of any electronic device (such as a television, a personal computer, a laptop computer, a mobile phone, a tablet computing device, or the like) which can be used in order to display images to a user. The display device receives a control signal from the control unit 3006 of apparatus 3000 which controls the display device to display an image on the display screen 6000. The display device may receive the image data for display as part of the image to be displayed on the display screen 6000 separately from the control signal; however, the present disclosure is not particularly limited in this regard.

In the example of FIG. 6A, the display device 6000 is configured to display first image data (being image data from a first image source) in region 6002 of the display device. Image data from a respective second, third and fourth image source are displayed in regions 6004, 6006 and 6008 of the display screen 6000 (based on the control signal which has been received from the control unit 3006 of apparatus 3000).

While image data from only four image sources are shown in the example of FIG. 6A, it will be appreciated that the present disclosure is not particularly limited in this regard. That is, image data from a number of image sources less than four or much greater than four may also be shown on the display screen based on the control signal which has been received from the control unit 3006 of apparatus 3000. Moreover, it will be appreciated that apparatus 3000 may optionally receive an image signal from a first number of image sources (such as ten image sources, for example) and then generate a control signal to display only image data from a selected portion of these image sources (such as the image data from the four image sources shown in FIG. 6A). The present disclosure is not particularly limited in this respect.

As such, in some examples, the control signal generated by control unit 3006 of apparatus 3000 is configured for controlling the display device to display data from number of image sources, wherein the number of image sources which form the plurality of image sources is determined in accordance with the assessment which has been generated for each of the image signals.

Now, in the example of FIG. 6A of the present disclosure, the first region 6002 in which image data from a first image source is displayed is a much bigger region of the display than the regions 6004, 6006 and 6008. This may be because apparatus 3000 has determined that the image source supplying the image data shown in the region 6002 is an active image source or a high priority image source, for example. Accordingly, a person viewing the display 6000 (such as a medical professional or the like) can easily identify the highest priority image and, furthermore, can obtain all the necessary information from this image because it is displayed in the region 6002.

In some examples, the control signal generated by control unit 3006 of apparatus 3000 is configured for controlling the display device to display the plurality of image sources in separate image panels, with a coloured outline on each panel determined in in accordance with the assessment which has been generated for each of the plurality of image signals. In the example of FIG. 6A of the present disclosure, for example, a coloured outline 6002A (such as an image boarder or the like) can be provided around one or more of the image regions 6002, 6004, 6006 and 6008. This coloured outline 6002A may be used in order to direct a person's attention to a specific image region. In some examples, the coloured outline 6002A may be provided around the highest priority image. Alternatively, the coloured outline may be provided around an active image. Alternatively, the coloured outline may be provided around an image when it changes state (e.g. transitions from a low priority image to a high priority image or from an inactive image to an active image). Alternatively, in some examples, coloured outlines with different properties (different thicknesses, different patterns, different colours or the like) may be used around images with different levels of priority or activity. For example, a red coloured outline may be provided around the most active image, while a yellow coloured outline may be provided around the second most active image (or around images which have a level of activity exceeding a predetermined activity threshold). The coloured outline may then be changed dynamically based on the detection result (i.e. as changes in the image data occur).

Consider an example whereby a coloured outline of a predetermined colour is provided around an image region in accordance with the level of activity of the image. In this situation, a medical professional viewing the image display can easily identify the most active images without performing a detailed assessment of the image. The most active images may be the images where something of importance is happening (such as increased level of activity during a critical stage of a surgical operation). Therefore, it is advantageous that medical staff can quickly identify the most active images, as remote medical support can then be provided to the areas in which it is most required.

In addition, by changing the thickness of the overlapped coloured outline in accordance with the priority of the image data, medical staff can quickly and easily understand the priority of the image from an image source and the activity of the image from an image source. This means that medical image staff can focus on the highest priority (e.g. most urgent) situations.

While a coloured outline is described with reference to FIG. 6A of the present disclosure, it will be appreciated that any other type of visual object (such as a marker, a box, an arrow or the like) which can be overlaid on or displayed with the respective image panels may be used in accordance with embodiments of the disclosure. Indeed, in some examples, control unit 3006 of apparatus 3000 may be configured to generate the control signal such that a caution or other type of warning is provided on a certain image panel. The caution may be generated when apparatus 3000 detects that a surgical operation has lasted for a long time, that new or additional staff are required, if the patient becomes unstable or the like. By providing this caution on the display, a person can rapidly identify a potentially high risk situation. This enables appropriate medical support to be provided as quickly as possible.

Consider, again, the example described with reference to FIG. 2A of the present disclosure (the hospital environment). When used in a medical environment such as this, it may be advantageous for image data from image sources in all operation rooms 2002 and all intensive care units 2006 (i.e. all rooms/beds) to be displayed on the display screen. This enables a person (such as a medical professional or the like) to monitor all regions of the medical environment. However, because the display displays the image data from the image sources in the manner described with reference to FIG. 6A of the present disclosure (based on the control signal which has been generated by control unit 3006 of apparatus 3000) the medical professional can quickly and easily identify the most relevant image data from amongst all of the image data shown on the display. Therefore, even when a large amount of image data is displayed on the display, the necessary information can be obtained quickly and easily.

Alternatively, in some examples, it may be advantageous in a medical environment to show image data only from active image sources. This reduces the amount of image data shown on the display (because image data from inactive image sources will not be shown on the display) ensuring that active panels can be seen more easily. The selection of active image sources may, in some examples, be dynamically updated such that the number of image sources from which image data is shown changes in accordance with the number of active image sources (i.e. the number of active rooms/beds). Thus, the limited available screen space is used more effectively in this situation. Moreover, because the display displays the image data from the image sources in the manner described with reference to FIG. 6A of the present disclosure (based on the control signal which has been generated by control unit 3006 of apparatus 3000) the medical professional can quickly and easily identify the most relevant image data from amongst the selection of image data shown on the display. Therefore, necessary information can be obtained more quickly and easily.

Compared with an image display such as that illustrated in FIG. 2B of the present disclosure, an image displayed based on the control signal generated by control unit 3006 of apparatus 3000 enables a medical professional to quickly and reliably receive necessary medical information, monitor the progress of an operation in an operating room and/or ascertain the status of an intensive care unit.

FIG. 6B illustrates an example image display in a medical environment in accordance with embodiments of the disclosure.

This example is similar to the example illustrated in FIG. 6A of the present disclosure. However, in this example, at least one of the size and layout of the image panels (image regions) in which image data from the image sources are displayed are changed in accordance with the assessment which has been generated for each of the plurality of image signals.

In this example, an image panel 6010, an image panel 6012, an image panel 6014 and an image panel 6016 are displayed on a display 6000. These panels are shown in a first configuration, where a highest priority image is displayed in the panel 6010 and a lowest priority image is displayed in image panel 6016.

Then, as the priority of the different images change, the image panel in which that image is displayed may be updated. For example, an image from an image source which was initially a high priority image may be initially displayed in image panel 6010. Then, when the priority of that image from that image source decreases, the image from that image source may be displayed in image panel 6012, 6014 or 6016 (lowest priority position). Therefore, a person (such as a medical professional or the like) can easily identify the priority of an image based on the location of that image on the display.

Alternatively, the size of the image panels may be changed (with a size of a panel increasing when image data of high priority is shown in that image panel). This enables a person (such as a medical professional or the like) to easily identify the priority of an image based on the size of that image on the display.

While the adaption of the layout or size of the image panels has been described with reference to the priority of the image, the present disclosure is not particularly limited in this regard. Alternatively, at least one of the size and location of the image panels may be changed in accordance with the activity of the image data. In fact, at least one of the size and location of the image data may be changed in accordance with the assessment which has been generated by apparatus 3000.

As such, according to embodiments of the disclosure, the control signal generated by control unit 3006 of apparatus 3000 is configured for controlling the display device to display the image sources in the separate image panels wherein at least one of the size of each image panel and the layout of the image panels is determined in accordance with the assessment which has been generated for each of the plurality of image signals

### <Advantageous Technical Effects>

In this way, the medical image processing device 3000 of the present disclosure may be configured to control the display of images from a plurality of image sources within a medical environment (such as hospital 2000 described with reference to FIG. 2A and FIG. 2B of the present disclosure). The medical image processing device 3000 efficiently optimizes the output from a plurality of image sources and may therefore enable a medical professional to quickly and reliably receive necessary medical information, monitor the progress of an operation in an operating room and/or ascertain the status of an intensive care unit.

As an example, a medical professional in a medical office 2010 such as that described with reference to FIG. 2 of the present disclosure, may be able to quickly and reliably receive necessary medical information regarding an operation being performed in a number of operating rooms 2002 located within a hospital 2000. Indeed, the medical processional may be able to easily identify the most active operation and/or the highest priority operation at any given time. This means that a higher level of remote support or instruction may be provided by the medical professional in the medical office 2010 to a surgeon performing an operation.

Of course, it will be appreciated that the present disclosure is not limited to these advantageous technical effects. Other advantageous technical effects will become apparent to the skilled person when reading the disclosure.

### <Method>

FIG. 7 illustrates an example method in accordance with embodiments of the disclosure. The example method of FIG. 7 may be performed by an apparatus such as apparatus 1000 or apparatus 3000 described with reference to FIGS. 1 and 3 of the present disclosure, for example.

The method of FIG. 7 starts at step S7000 and proceeds to step S7002.

In step S7002, the method comprises receiving a plurality of image signals from a respective plurality of image sources, the plurality of image signals each including image data for display and additional data, wherein the image data and/or the additional data includes information regarding one or more vital signs of a patient being the target of that image source.

Once the image signals have been received in step S7002, the method proceeds to step S7004.

In step S7004, the method comprises generating an assessment for each of the plurality of image signals in accordance with processing performed on the image data and/or the additional data of each image signal.

Once the assessment for each of the plurality of image signals has been generated in step S7004, the method proceeds to step S7006.

In step S7006, the method comprises generating a control signal for controlling a display device to display an image comprising image data of at least one of the image signals from the plurality of image sources in accordance with the assessment which has been generated for each of the plurality of image signals.

The method than proceeds to, and ends with, step S7008.

In this way, the method described with reference to FIG. 7 of the present disclosure enables the output from a plurality of image sources to be efficiently optimized. Therefore a medical professional may quickly and reliably receive necessary medical information, monitor the progress of an operation in an operating room and/or ascertain the status of an intensive care unit.

It will be appreciated that the embodiments of the present disclosure are not particularly limited to the order to the method steps described with reference to FIG. 7 of the present disclosure. A number of the different steps of the method may be performed in a different order. Alternatively, the method steps may be performed in parallel. For example, the step of generating the assessment for the plurality of image signals may be performed as the image signals are being received (even before all image signals have been received). This enables the method to be more efficiently performed. Moreover, it enables the method to dynamically adapt the control signal to control the display device to display an image as new image signals are received and/or as the content of those image signals changes (e.g. as a given image source becomes active and/or a change in priority of the image signal occurs).

In addition, embodiments of the present disclosure may be arranged in accordance with the following numbered clauses:

1. A medical image processing device for controlling display of an image from a plurality of image sources, the medical image processing device comprising processing circuitry configured to:
receive a plurality of image signals from a respective plurality of image sources, the plurality of image signals each including image data for display and additional data, wherein at least one the image data and the additional data includes information regarding one or more vital signs of a patient being the target of that image source;
generate an assessment for each of the plurality of image signals in accordance with processing performed on at least one of the image data and the additional data of each image signal; and
generate a control signal for controlling a display device to display an image comprising image data of at least one of the image signals from the plurality of image sources in accordance with the assessment which has been generated for each of the plurality of image signals.

2. The medical image processing device according to clause 1, wherein the processing performed on the at least one of the image data and the additional data of each image signal is processing to determine whether the image signal from each image source is active or inactive.

3. The medical image processing device according to clause 2, wherein the processing circuitry is configured to determine that an image signal from an image source is inactive if the image data of the image signal does not contain any image information.

4. The medical image processing device according to clause 2, wherein the processing to determine whether the image signal from each image source is active or inactive is based on at least one of a level of motion detected in the image data, a type of an action detected in the image data, a type of person detected in the image data, information regarding a noise level determined from the additional data, schedule information determined from the additional data and the vital signs of a patient being the target of that image source.

5. The medical image processing device according to clause 2, wherein the control signal is configured for controlling the display device to display an image comprising image data from a given image source when the image signal from that image source is active.

6. The medical image processing device according to any preceding clause, wherein the processing performed on the at least one of the image data and the additional data of each image signal is processing to determine the priority of the image signal from each image source.

7. The medical image processing device according to clause 6, wherein the priority of the image signal from each image source is determined based on the information regarding one or more vital signs of the patient being the target of that image source.

8. The medical image processing device according to clause 7, wherein the vital signs of the patient include at least one of the body temperature of the patient, the pulse rate of the patient, the respiration rate of the patient and the blood pressure of the patient.

9. The medical image processing device according to clause 6, wherein the priority of the image signal from each image source located in an operation room is determined in accordance with at least one of a level of skill of medical staff performing a medical operation, a level of experience of medical staff performing a medical operation, a type of surgery being performed and a duration of a surgery being performed.

10. The medical image processing device according to clause 6, wherein the priority of the image signal from each image source located in an intensive care unit is determined in accordance with at least one of whether a bed is used or not, a level of skill of medical staff caring for the patient, a level of experience of medical staff caring for the patient, a characteristic of the patient, a type of disease suffered by the patient, and a type of injury suffered by the patient.

11. The medical image processing device according to any preceding clause, wherein the control signal is configured for controlling a display device to display an image comprising image data from a plurality of image sources in accordance with the assessment which has been generated for each of the plurality of image sources.

12. The medical image processing device according to clause 11, wherein the control signal is configured for controlling the display device to display the plurality of image sources in separate image panels, with a coloured outline on each panel determined in in accordance with the assessment which has been generated for each of the plurality of image signals.

13. The medical image processing device according to clause 12, wherein the control signal is configured for controlling the display device to display the image sources in the separate image panels wherein at least one of the size of each image panel and the layout of the image panels is determined in accordance with the assessment which has been generated for each of the plurality of image signals.

14. The medical image processing device according to any preceding clause, wherein the control signal is configured for controlling the display device to display data from number of image sources, wherein the number of image sources which form the plurality of image sources is determined in accordance with the assessment which has been generated for each of the image signals.

15. The medical image processing device according to any preceding clause, wherein the processing performed on the at least one of the image data and the additional data of each image signal to generate an assessment for each of the plurality of image signals includes use of a machine learning or neural network model.

16. The medical image processing device according to any preceding clause, wherein the assessment for each the plurality of images includes a score determined based on at least one of whether the image signal from each image source is active or inactive and the priority of the image signal from each image source.

17. A medical image processing method of controlling display of an image from a plurality of image sources, the method comprising:
receiving a plurality of image signals from a respective plurality of image sources, the plurality of image signals each including image data for display and additional data, wherein at least one of the image data and the additional data includes information regarding one or more vital signs of a patient being the target of that image source;
generating an assessment for each of the plurality of image signals in accordance with processing performed on the at least one of the image data and the additional data of each image signal; and
generating a control signal for controlling a display device to display an image comprising image data of at least one of the image signals from the plurality of image sources in accordance with the assessment which has been generated for each of the plurality of image signals.

18. A computer program product comprising instructions which, when the instructions are implemented by a computer, cause the computer to perform a medical image processing method of controlling display of an image from a plurality of image sources, the method comprising:
receiving a plurality of image signals from a respective plurality of image sources, the plurality of image signals each including image data for display and additional data, wherein at least one of the image data and the additional data includes information regarding one or more vital signs of a patient being the target of that image source;
generating an assessment for each of the plurality of image signals in accordance with processing performed on the at least one of the image data and the additional data of each image signal; and
generating a control signal for controlling a display device to display an image comprising image data of at least one of the image signals from the plurality of image sources in accordance with the assessment which has been generated for each of the plurality of image signals.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A medical image processing device for controlling display of an image from a plurality of image sources, the medical image processing device comprising processing circuitry configured to:
receive a plurality of image signals from a respective plurality of image sources, the plurality of image signals each including image data for display and additional data, wherein at least one the image data and the additional data includes information regarding one or more vital signs of a patient being the target of that image source;
generate an assessment for each of the plurality of image signals in accordance with processing performed on at least one of the image data and the additional data of each image signal; and
generate a control signal for controlling a display device to display an image comprising image data of at least one of the image signals from the plurality of image sources in accordance with the assessment which has been generated for each of the plurality of image signals.

2. The medical image processing device according to claim 1, wherein the processing performed on the at least one of the image data and the additional data of each image signal is processing to determine whether the image signal from each image source is active or inactive.

3. The medical image processing device according to claim 2, wherein the processing circuitry is configured to determine that an image signal from an image source is inactive if the image data of the image signal does not contain any image information.

4. The medical image processing device according to claim 2, wherein the processing to determine whether the image signal from each image source is active or inactive is based on at least one of a level of motion detected in the image data, a type of an action detected in the image data, a type of person detected in the image data, information regarding a noise level determined from the additional data, schedule information determined from the additional data and the vital signs of a patient being the target of that image source.

5. The medical image processing device according to claim 2, wherein the control signal is configured for controlling the display device to display an image comprising image data from a given image source when the image signal from that image source is active.

6. The medical image processing device according to claim 1, wherein the processing performed on the at least one of the image data and the additional data of each image signal is processing to determine the priority of the image signal from each image source.

7. The medical image processing device according to claim 6, wherein the priority of the image signal from each image source is determined based on the information regarding one or more vital signs of the patient being the target of that image source.

8. The medical image processing device according to claim 7, wherein the vital signs of the patient include at least one of the body temperature of the patient, the pulse rate of the patient, the respiration rate of the patient and the blood pressure of the patient.

9. The medical image processing device according to claim 6, wherein the priority of the image signal from each image source located in an operation room is determined in accordance with at least one of a level of skill of medical staff performing a medical operation, a level of experience of medical staff performing a medical operation, a type of surgery being performed and a duration of a surgery being performed.

10. The medical image processing device according to claim 6, wherein the priority of the image signal from each image source located in an intensive care unit is determined in accordance with at least one of whether a bed is used or not, a level of skill of medical staff caring for the patient, a level of experience of medical staff caring for the patient, a characteristic of the patient, a type of disease suffered by the patient, and a type of injury suffered by the patient.

11. The medical image processing device according to claim 1, wherein the control signal is configured for controlling a display device to display an image comprising image data from a plurality of image sources in accordance with the assessment which has been generated for each of the plurality of image sources.

12. The medical image processing device according to claim 11, wherein the control signal is configured for controlling the display device to display the plurality of image sources in separate image panels, with a coloured outline on each panel determined in in accordance with the assessment which has been generated for each of the plurality of image signals.

13. The medical image processing device according to claim 12, wherein the control signal is configured for controlling the display device to display the image sources in the separate image panels wherein at least one of the size of each image panel and the layout of the image panels is determined in accordance with the assessment which has been generated for each of the plurality of image signals.

14. The medical image processing device according to claim 1, wherein the control signal is configured for controlling the display device to display data from number of image sources, wherein the number of image sources which form the plurality of image sources is determined in accordance with the assessment which has been generated for each of the image signals.

15. The medical image processing device according to claim 1, wherein the processing performed on the at least one of the image data and the additional data of each image signal to generate an assessment for each of the plurality of image signals includes use of a machine learning or neural network model.

16. The medical image processing device according to claim 1, wherein the assessment for each the plurality of images includes a score determined based on at least one of whether the image signal from each image source is active or inactive and the priority of the image signal from each image source.

17. A medical image processing method of controlling display of an image from a plurality of image sources, the method comprising:
receiving a plurality of image signals from a respective plurality of image sources, the plurality of image signals each including image data for display and additional data, wherein at least one of the image data and the additional data includes information regarding one or more vital signs of a patient being the target of that image source;
generating an assessment for each of the plurality of image signals in accordance with processing performed on the at least one of the image data and the additional data of each image signal; and
generating a control signal for controlling a display device to display an image comprising image data of at least one of the image signals from the plurality of image sources in accordance with the assessment which has been generated for each of the plurality of image signals.

18. A non-transitory computer readable medium storing a computer program product comprising instructions which, when the instructions are implemented by a computer, cause the computer to perform a medical image processing method of controlling display of an image from a plurality of image sources, the method comprising:
receiving a plurality of image signals from a respective plurality of image sources, the plurality of image signals each including image data for display and additional data, wherein at least one of the image data and the additional data includes information regarding one or more vital signs of a patient being the target of that image source;
generating an assessment for each of the plurality of image signals in accordance with processing performed on the at least one of the image data and the additional data of each image signal; and
generating a control signal for controlling a display device to display an image comprising image data of at least one of the image signals from the plurality of image sources in accordance with the assessment which has been generated for each of the plurality of image signals.
